(19) United States
(12) Patent Application Publication
(10) Pub. No.: US 2011/0125097 A1
(43) Pub. Date: May 26, 2011

(54) Catheter Introducer with Hub Seal and Removal Tab

(76) Inventors: Thomas J. Shaw, Frisco, TX (US); Ni Zhu, Plano, TX (US); Kathryn Margaret Duesman, Pilot Point, TX (US)

(21) Appl. No.: 12/624,962

(22) Filed: Nov. 24, 2009

(51) Int. Cl. A61M 25/06

(52) U.S. Cl. 604/164.12; 604/167.01

(57) ABSTRACT

A catheter introducer having a catheter hub seal that is engageable with the rear opening of the catheter hub to impede liquid flow out of the catheter hub following catheter insertion and subsequent withdrawal of the needle following separation of the housing from the catheter hub. The catheter hub seal is preferably made of a rubbery material and can be disposed between the housing and catheter assembly or can be attachable to the catheter assembly.

### BACKGROUND OF THE INVENTION

1. Field of the Invention

The invention is a medical device that is used to introduce a catheter into a patient's body, and relates more particularly to a catheter introducer used to obtain access to blood vessels. One aspect of the invention relates to a catheter introducer having a catheter hub seal, which can be made of a rubbery material, that reduces the leakage of body liquids, such as blood, out the rear opening of a catheter hub following insertion of the catheter and separation of the housing and hub following withdrawal of the needle used during insertion. Another aspect of the invention relates to an intravenous (“IV”) catheter having an elastomeric hub seal that protects the patient against external contaminants that can otherwise enter a patient's blood vessels through the rear opening of a catheter hub following insertion of the catheter and removal of a needle used during insertion. The needle can be withdrawn manually, through use of a biasing or retraction mechanism, or by a combination of manual force and a biasing force. Where the needle is retractable inside the housing of the catheter introducer following insertion, the subject invention also reduces the likelihood of needlestick injuries and related pathogenic contamination to medical personnel and others. Still another aspect of the invention relates to a tab that facilitates removal of the elastomeric hub seal from the rear opening of the catheter hub.

2. Description of Related Art

Catheters and IV catheter insertion devices are well known. When a catheter is inserted into a patient to access blood vessels, a disposable needle extending through the catheter punctures the vessel and permits insertion of a flexible cannula into the vessel. The needle is then withdrawn from the vessel through the cannula and hub, leaving the cannula in place for blood vessel access either continuously (as for infusion) or intermittently.

In recent years, because of the prevalence of blood-borne pathogens such as HIV and hepatitis, there has been an increasing need for IV catheter introducers that prevent accidental needlestick injuries to medical personnel and to other employees who handle trash, laundry or other refuse containing used needles. As a result, new products have been designed that incorporate special needle covers or mechanisms for retracting the needle into the device housing following use. Such devices are disclosed, for example, in U.S. Pat. Nos. 4,747,831; 4,828,548; 5,129,884; 5,501,675; 5,746,215; 5,817,058; 5,989,220; 6,083,202; 6,090,078; and 6,096,005. Some of the prior art devices contain numerous complicated parts that substantially increase manufacturing costs and interfere with the user's ability to feel when the needle is properly inserted into the patient. Other devices are positionally unstable, require two-handed operation or are prone to premature needle retraction during shipment, storage and handling. More recently, an IV catheter introducer has been disclosed in U.S. Pat. No. 6,872,193 that embodies some additional benefits and advantages.

Applicants have now discovered a new IV catheter introducer embodying additional structural elements providing more advantages over the devices disclosed in the prior art.

### SUMMARY OF THE INVENTION

The present invention is a catheter introducer that comprises a catheter hub seal optionally having a removal tab. The hub seal provides many advantages over prior art catheter introducers, including, for example, reducing the leakage of body liquids, such as blood, out the rear opening of a catheter hub following insertion of the catheter cannula and withdrawal of the needle. Catheter introducers embodying the invention preferably comprise a retraction mechanism for withdrawing the needle into the housing and plunger assembly, but can also embody needles that are not retractable into the device following use.

One embodiment of the invention comprises a catheter hub seal that is located between the housing and catheter assembly during catheter insertion and remains engaged with the hub following needle withdrawal. In this embodiment, the catheter hub seal can help stabilize the catheter hub during advancement of the needle and catheter into a blood vessel. As the needle is subsequently withdrawn from the catheter cannula, a thin web section of the catheter hub seal can wipe the needle and then close to reduce the likelihood of blood spillage out the back of the hub. The hub seal can later be removed from the rear opening of the hub prior to accessing the blood vessel into which the catheter cannula has previously been inserted. The optional removal tab provides the user with an easily gripped means of removing the catheter hub seal from the back of the hub.

Another embodiment of the invention comprises a catheter hub seal, which can be made of an elastomeric material, that is either attached or attachable to the catheter assembly and is thereby readily available for insertion into the rear opening of the catheter hub following withdrawal of the needle from the catheter cannula and hub. With this embodiment of the invention, the hub seal is not penetrated by the needle. One suitable attachment means is a stretchable attachment ring that frictionally engages the hub and has a unitarily molded, foldable hinge connecting the attachment ring to a hub seal and removal tab. It will be appreciate upon reading this disclosure, however, that other similarly effective attachment means such as, for example, a deformable split ring can be used in place of the hub seal attachment ring as shown in the drawings.

According to another embodiment of the invention, an IV catheter introducer is disclosed that comprises an elastomeric hub seal that reduces the likelihood of introducing external contaminants into a patient's blood vessels through the rear opening of a catheter hub following insertion of the cannula into a blood vessel, removal of the needle and disengagement of the plunger assembly and housing from the catheter hub.

According to another embodiment of the invention, an IV catheter introducer is disclosed that comprises an elastomeric catheter hub seal and a tab useful for removing the elastomeric hub seal from the rear of the catheter hub prior to connecting the hub to another device. When the catheter hub seal is provided with an outwardly projecting tab, the tab also serves as a quick visual indicator as to the orientation of the beveled side of the needle. If desired, the tab can be positionally aligned with the beveled side of the needle on the “top” side of the device.

According to another embodiment of the invention, an IV-catheter introducer is disclosed that comprises a housing, a plunger and a needle assembly insertable into the rear of the housing, a catheter hub releasably attachable to the front of the housing, and an elastomeric hub seal with a pierceable web disposed between the housing and the catheter hub. When used with a retractable needle, the pierceable web of the elastomeric hub seal also wipes the needle during retraction and dampens the retraction force to reduce the likelihood of forward splattering of blood during retraction. When the needle is retractable inside the housing of the catheter introducer following insertion of the cannula, the subject invention reduces the likelihood of needle stick injuries and related pathogenic contamination to medical personnel and others. In one embodiment of the invention, the elastomeric hub seal can also facilitate attachment of the housing to the catheter hub and can stabilize the catheter hub during advancement of the needle and catheter into the blood vessel.

According to another embodiment of the invention, a rearwardly facing projection is provided on the back side of the elastomeric hub seal to facilitate rotational alignment of the hub seal with a notch disposed in the perimeter of a forwardly facing flange of the housing.

According to another embodiment of the invention, an IV catheter introducer is disclosed that comprises a housing, a plunger and retractable needle assembly insertable into the rear of the housing, a catheter hub releasably attachable to the front of the housing, and an elastomeric hub seal attachable to the catheter hub, said catheter hub seal being linked or tethered to the hub prior to use and being insertable into the rear opening of the catheter hub following retraction of the needle and separation of the housing from the catheter hub. With this embodiment, as before, a tab can be provided as part of the elastomeric hub seal to facilitate subsequent removal of the catheter hub seal from the catheter hub to facilitate connection of the catheter hub to another device.

According to another embodiment of the invention, the IV catheter introducer of the invention as disclosed herein can be used in combination with a catheter hub that is ported or that comprises transversely projecting wings.

### BRIEF DESCRIPTION OF THE DRAWINGS

The apparatus of the invention is further described and explained in relation to the following figures of the drawing wherein:

FIG. 1 is a simplified perspective view of an IV catheter introducer as disclosed herein;

FIG. 2 is an exploded perspective view of the IV catheter introducer of FIG. 1;

FIG. 3 is a front elevation view, partially broken away, of the IV catheter introducer of FIG. 1;

FIG. 4 is a left side view of the IV catheter introducer of FIG. 3;

FIG. 5 is a right side view of the IV catheter introducer of FIG. 3;

FIG. 6 is a top plan view of the IV catheter introducer of FIG. 3;

FIG. 7 is a cross-sectional view taken along line 7-7 of FIG. 4;

FIG. 8 is an enlarged, detail view of part of the IV catheter introducer of FIG. 7;

FIG. 9 is a rotated front elevation view of another IV catheter introducer as disclosed herein (without a needle cover);

FIG. 10 is an exploded perspective view of the IV catheter introducer of FIG. 9;

FIG. 11 is an enlarged cross-sectional view taken along line 11-11 of FIG. 9;

FIG. 12 is an enlarged detail front elevation view of a catheter hub, cannula and foldable catheter hub seal in an unfolded position;

FIG. 13 is an enlarged detail view as in FIG. 12 but with the foldable catheter hub seal shown in a folded, sealing position;

FIG. 14 is a left side elevation view of the structure of FIG. 13;

FIG. 15 is a right side elevation view of the structure of FIG. 13; and

FIG. 16 is an enlarged cross-sectional view showing the device of FIG. 11 following retraction of the needle.

### DETAILED DESCRIPTION

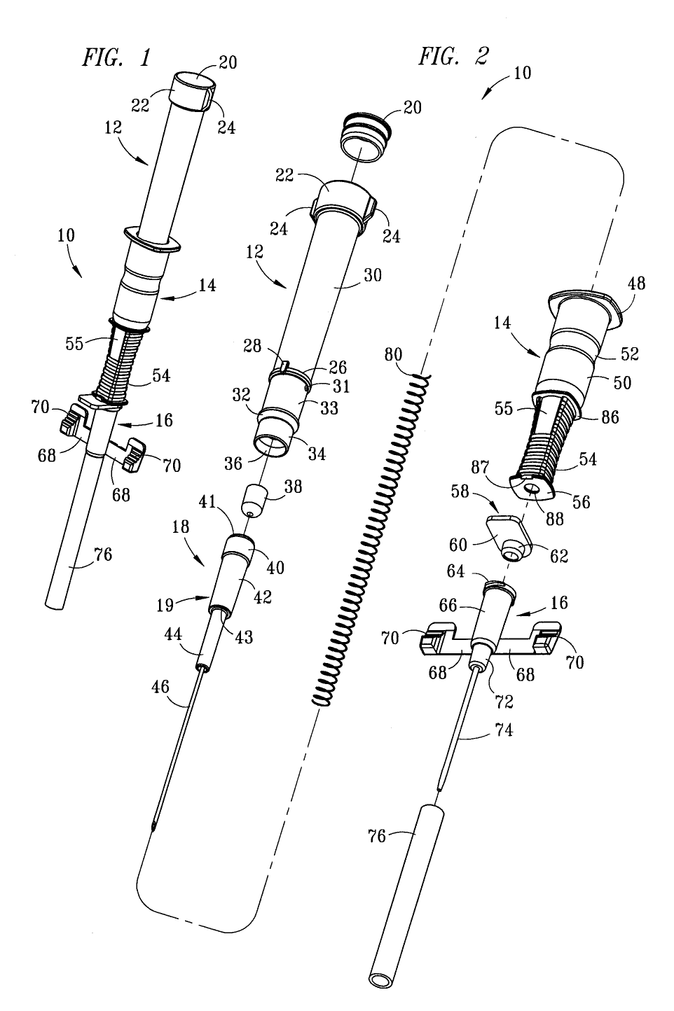

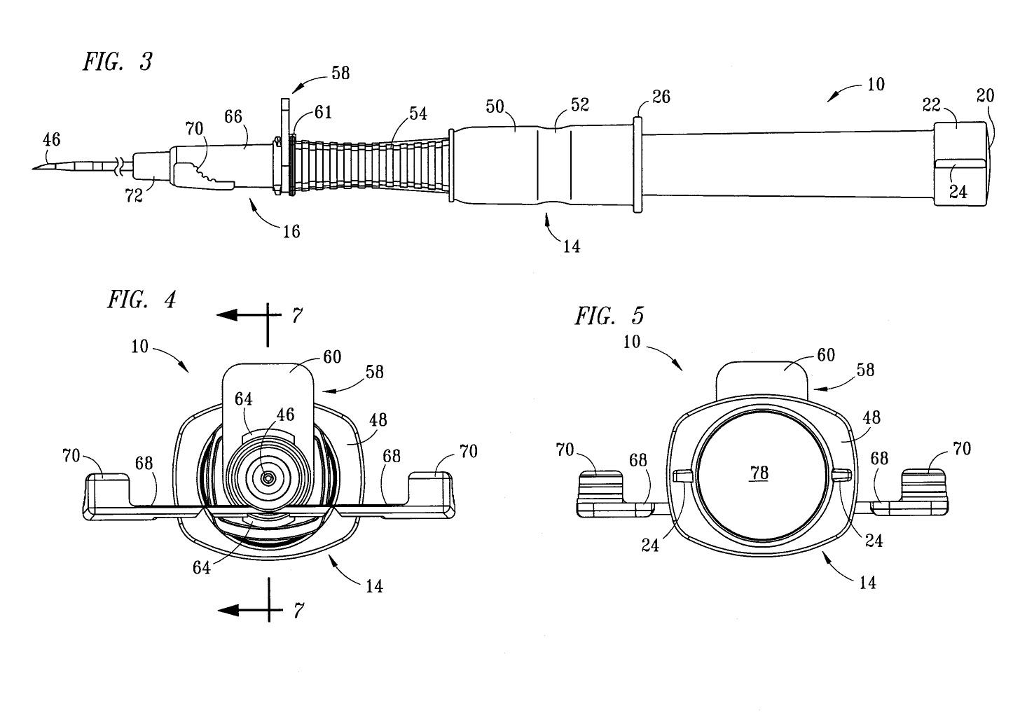

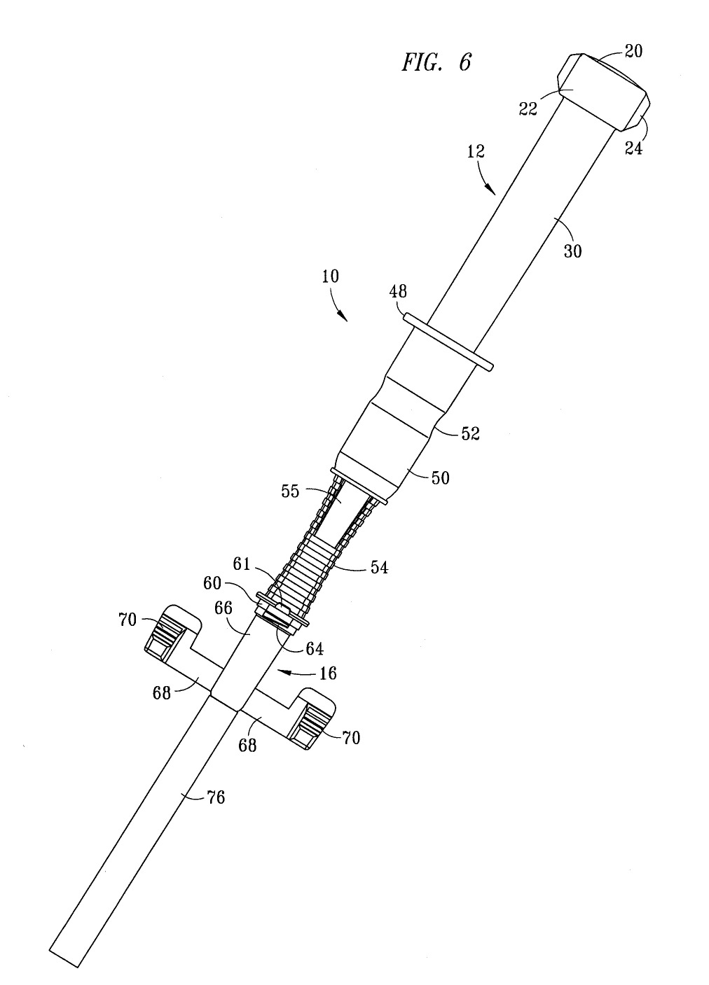

Referring to the embodiment of FIGS. 1-8, one IV catheter introducer 10 of the invention comprises plunger assembly 12, housing 14, needle assembly 18, retraction spring 80, catheter hub assembly 16, catheter hub seal 58 disposed between housing 14 and catheter hub assembly 16, and removable cover 76. Although a retractable needle is not required for use of catheter hub seal 58 of the invention, the use of a retractable needle reduces the likelihood of inadvertent needle stick injuries once housing 14 is separated from catheter hub 16 and needle 46 is withdrawn from flexible cannula 74. Plunger assembly 12, housing 14, the body portions of retractable needle assembly 18, catheter hub assembly 16 and cover 76 can be made of FDA-approved, moldable polymeric materials, although it will be understood that different parts can comprise different materials. Catheter hub seal 58 can be made of a polymeric material, and most preferably, an elastomeric material such as rubber.

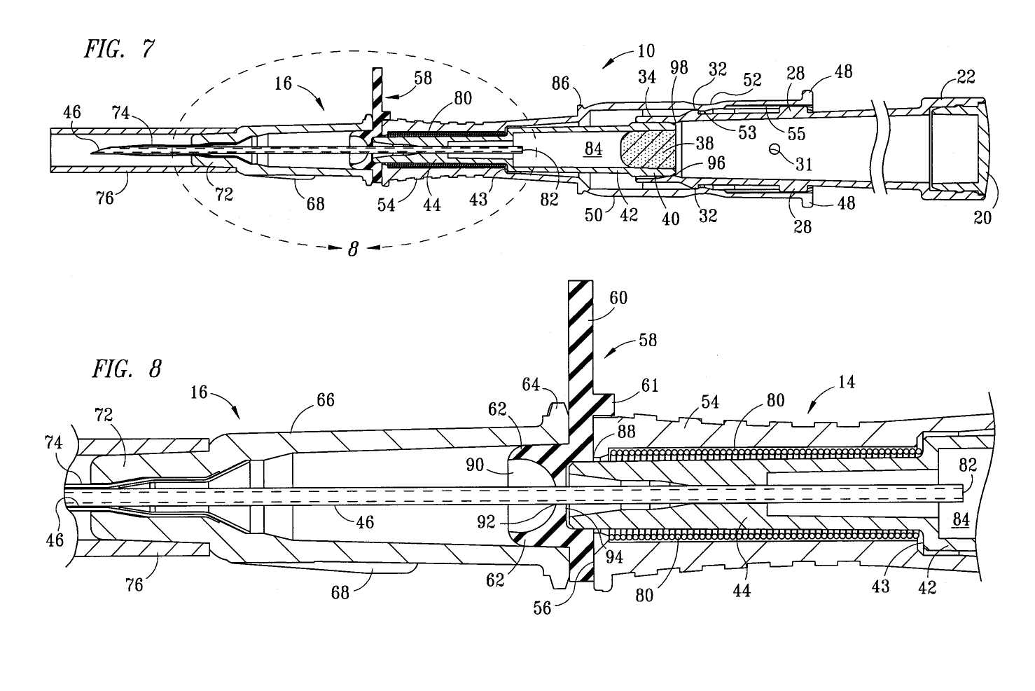

Plunger assembly 12 comprises a substantially tubular body with three body sections 30, 33, 34. Plunger body sections 30, 33 have an inside diameter that tapers inwardly from rear to front, and plunger body section 30 has a rear opening surrounded by collar 22. End cap 20 is desirably insertable into frictional engagement with the inside of collar 22 (more visible in FIG. 7), although other similarly effective cap configurations can likewise be used. The forwardly extending, smaller-diameter front section 34 of plunger assembly 12 can comprise an opening 36 having a length and inside diameter sufficient to receive rearwardly facing end collar 40 of needle assembly 18. Vent hole 31 (FIG. 7) can be provided in plunger body 30 to reduce the likelihood of blood or other body fluid splattering forwardly out of IV catheter introducer 10 during retraction.

Needle assembly 18 comprises needle holder 19 and needle 46. Needle holder 19 further comprises end collar 40 and forwardly extending, inwardly stepped sections 42 and 44 separated by annular shoulder 43. Section 44 of needle holder 19 comprises a coaxial longitudinal passageway sized to receive and hold a portion of the shaft of needle 46 in a fixed axial position relative to needle holder 19. Section 44 of needle holder 19 also has an outside diameter small enough to be insertable into coiled retraction spring 80. Annular shoulder 43 has sufficient width to abut the top of retraction spring 80 when installed inside housing 14 and to limit the rearward expansion of spring 80 prior to retraction.

Needle 46 has a beveled front end, a blunt rear end, with the blunt end being insertable into, and held in fixed relation to, section 44 of needle holder 19 by any suitable means, preferably by a commercially available adhesive satisfactory for use in such applications. Such adhesive can be laser cured to facilitate high-speed manufacture. Needle 46 as shown is long enough to project forwardly from section 44 a distance sufficient to be clinically effective for insertion through catheter hub assembly 16, and to project rearwardly through the interior of sections 44, 42 and collar 40 so that blunt end 82 extends into flash chamber 84 (FIGS. 7 and 8) inside body section 42.

Needle holder 19 can be unitarily molded out of substantially transparent plastic to facilitate viewing of liquid, such as blood, inside flash chamber 84 through window 55 in housing 14 during use of the device. This serves as a visual indicator to the user that needle 46 and catheter cannula 74 have been successfully inserted into a vessel. End collar 40 of needle holder 19 can have an outside diameter sized to slidably engage the inside surface of front section 34 of plunger assembly 12 and an inside surface sized to frictionally engage plug 38.

During assembly of catheter introducer 10, plug 38 can be installed inside collar 40 prior to inserting collar 40 into front section 34 of the plunger assembly. A principal function of plug 38 is to retain a bodily liquid, such as blood, that enters flash chamber 84 inside stepped body section 42 of needle holder 19 after insertion of needle 46 and catheter cannula 74 into a vessel and prior to withdrawal of needle 46 from cannula 74. Plug 38 can be made of a porous material capable of absorbing at least some of said liquid while preventing said liquid from flowing rearwardly out of needle holder 19. A shallow annular groove 41 (FIG. 2) provided at the rear of collar 40 cooperates with a cooperatively sized annular projection 98 (FIG. 7) inside front section 34 to assist in positioning collar 40 inside front section 34. Following joinder of needle assembly 18 to plunger assembly 12, they can be inserted as a single unit into housing 14 of catheter introducer 10. Referring to FIGS. 7 and 8, when catheter introducer 10 is fully assembled,

Housing 14 can comprise a centrally disposed, longitudinally extending tubular cavity that is coaxially aligned with the centrally disposed longitudinal cavities through plunger assembly 12, needle assembly 18, and catheter hub assembly 16. The inside diameter is stepped inwardly between flange 86 (FIG. 8) and front flange 56 to provide an annular seating surface for the forwardly extending end of spring 80 and an annular seating surface for annular shoulder 43 of needle assembly 18 inside housing 14 (as seen in FIG. 7). Rear flange 48, flange 86 and front flange 56 can be molded in a configuration (such as, for example, a modified rectangular configuration) to provide a substantially flat base for housing 14 when lying on a support surface. Referring to FIGS. 2 and 7, front flange 56 can comprise an opening 88 having an inside diameter slightly greater than the outside diameter of section 44 of needle assembly 18 to permit the forwardly extending tip of section 44 to pass through opening 88 for insertion into recess 94 in the back side of catheter hub seal and removal tab 58. Molded ridges 54 or other similarly effective surface texturing can be provided for use as finger grips and substantially flat wall section 55 can be provided as a viewing window into underlying flash chamber 84 of needle holder 19. Housing 14 and needle holder 19 can both be molded from clear plastic to facilitate viewing of blood flowing into flash chamber 84 from the blunt end of needle 46. One suitable, substantially clear plastic is polycarbonate.

Plunger assembly 12 can comprise annular rib 26 and circumferentially spaced, elongate keys 28 projecting radially outward from the outside surface of section 30 to guide plunger assembly 12 as it advances forwardly into housing 14 during assembly. Keys 28 can be alignable with longitudinally directed keyways 55 (FIG. 7) in the inside wall of housing 14 to maintain a predetermined rotational alignment of plunger assembly 12 and needle assembly 18 inside housing 14. This rotational alignment insures that the bevel of needle 46 is always upwardly facing as plunger assembly 12 is advanced forwardly relative to housing 14 during insertion of needle 46 into a vessel.

Referring again to FIGS. 2 and 7, forwardly facing inclined ramp 32 having a rearwardly facing, substantially square shoulder is provided at the rear of section 34 and between sections 34 and 33 of plunger assembly 12. Ramp 32 slides past inwardly projecting annular rib 53 of housing 14 when plunger assembly 12 is inserted into housing 14 during assembly of IV catheter introducer 10. Inwardly projecting annular rib 53 is provided inwardly of annular groove 52 in outside wall 50 of housing 14. Abutting engagement between the rearwardly facing shoulder of ramp 32 and annular rib 53 subsequently limits rearward movement of plunger body 30 relative to housing 14 sufficiently to prevent the forward end of the plunger from being accidentally or intentionally withdrawn from housing 14 prior to or following use of catheter introducer 10. Prior to use, the abutting engagement also resists the rearwardly directed force of compressed spring 80 pushing against annular shoulder 43 of needle assembly 18, which force is transmitted to plunger assembly 12 from needle assembly 18 through the engagement between collar 40 and the inside wall of section 34 in cooperation with inwardly facing annular rib 98 inside plunger assembly 12 (FIG. 7). The inside diameter of that portion of housing 14 that is opposite section 44 of needle holder 19 can be slightly greater than the outside diameter of spring 80 when compressed to accommodate the spring and to serve as an outer spring guide during compression as needle assembly 18 is seated inside housing 14 when ramp 32 of plunger assembly 12 is moved forwardly of annular inside rib 53 of housing 14.

According to one embodiment of the invention, catheter hub seal 58, best seen in FIGS. 2 and 8, is made of rubber and further comprises a substantially cylindrical, forwardly facing projection 62 having a forwardly facing concave arcuate recess 90, a rearwardly facing recess 94, and an upwardly projecting removal tab 60. Rearwardly facing recess 94 can be cylindrical or otherwise configured to receive the forwardly extending tip of body section 44 of retractable needle assembly 18. Removal tab 60, can face upwardly to quickly identify the top side of catheter introducer 10 (with the beveled point of needle 46 facing up), and a rearwardly projecting positioning tab 61 is insertable into notch 87 (FIG. 2) or another similarly effective recess in front flange 56 of housing 14 to position catheter hub seal 58 rotationally with respect to housing 14. Forwardly facing projection 62 can have an outside diameter that is sized to frictionally engage the inside wall of hub 66 of catheter hub assembly 16. Catheter hub seal 58 can comprise a thin elastomeric web (for example, about 0.020 inches in thickness) disposed between recesses 90, 94. During assembly of catheter introducer 10, aperture 92 can be formed by insertion of needle 46 through the web. Aperture 92 can lightly engage the outside surface of needle 46 without constricting against needle 46 sufficiently to prevent full retraction of the needle by spring 80 following insertion of catheter cannula 74 into a vessel.

Referring to FIGS. 1-8, catheter hub assembly 16 can comprise tapered tubular hub 66 having a connector collar 64 on the rearwardly facing end. Connector collar 64 can be configured for easy attachment to cooperatively configured end connectors of conventional, commercially available IV infusion sets, blood collection bags, or other devices or accessories. A pair of diametrically opposed, laterally extending wings 68 can be attached to, or unitarily molded as part of, hub 66 to provide lateral support for catheter hub assembly 16 when placed in service on the body of a patient. Textured finger pads 70 can be provided at or near the ends of wings 68 for use in stabilizing IV catheter assembly 16 during removal of catheter hub seal 58 from hub 66 of catheter introducer 10 at a time subsequent to withdrawal of needle 46 and separation of housing 14. Catheter cannula 74 can be made of a medically approved, flexible polymeric material, the cannula having an inside diameter slightly greater than the outside diameter of needle 46, and a length sufficient to cover all but the beveled end of needle 46 when needle 46 is inserted through the catheter cannula. Catheter cannula 74 can be secured to the inside of tapered, forwardly extending tip 72 of catheter hub assembly 16 by any suitable means such as a press-fit ring disposed between the hub and catheter, adhesive, laser or ultrasonic welding, or the like. Referring to FIGS. 1, 2, 7 and 8, slidable cover 76, can be made of molded plastic with a length sufficient to protect the beveled tip of needle 46 prior to use and with an inside diameter that frictionally engages the outside surface of tip 72 of catheter hub assembly 16.

Following assembly of catheter introducer 10, needle 46 projects forwardly from catheter cannula 74. The inside of hub 66 frictionally engages the outside surface of cylindrical projection 62 of catheter hub seal 58. The forwardly extending end of section 44 of needle assembly 18 (FIG. 2) can extend into and frictionally engage the circumferentially extending sidewall of recess 94 of catheter hub seal 58. Needle 46 forms and passes through aperture 92 in the thin web section of catheter hub seal 58 and is disposed in fixed longitudinal relation to section 44. Blunt end 82 of needle 46 desirably extends into flash chamber 84, which is visible through housing 14. Rear collar 40 of the needle holder is frictionally engaged by the inside surface of section 34 of plunger assembly 12, and the escape of body liquid from the rear of flash chamber 84 is blocked by frictionally engaged plug 38. Retraction spring 80 is compressed inside the longitudinally extending annular space between the back side of flange 56 of housing 14 and annular shoulder 43 of the needle holder, and is held in compression prior to retraction by contacting engagement between the square annular shoulder behind annular ramp 32 of plunger assembly 12 (FIG. 2) and inwardly facing annular ridge 53 of housing 14, and by contacting engagement between inwardly facing annular ridge 96 inside plunger assembly 12 and cooperating groove 98 in the rearwardly extending end portion 41 of collar 40. Although retraction spring 80 is depicted as fully compressed in FIGS. 7 and 8, it should be appreciated that the annular space into which spring 80 is compressed is desirably slightly longer than the fully compressed length of spring 80 to provide a slight tolerance during manufacture and assembly so that complete compression is not required in order for the spring to fit within the annular space.

Referring to FIGS. 1, 2, 7 and 8, following removal of cover 76, needle 46 and the forwardly extending tip of catheter 74 can be inserted into the body, such as into a blood vessel, by lightly gripping ribs 54 of housing 14 and applying forward pressure to housing 14 to advance needle 46 and catheter cannula 74 with the bevel of needle 46 facing upwardly. Once needle 46 punctures the wall of the blood vessel and catheter cannula 74 is properly positioned a sufficient distance inside the vessel, housing 14 can be separated a short distance, such as from about 0.25 to about 0.5 inches, from catheter hub seal 58 by applying downward pressure to wings 68 with one hand while pulling the housing gently backwards with the other hand until separation is achieved. During this time needle 46 remains inserted through hub seal 58 but the beveled end of needle 46 is withdrawn out of the patient's blood vessel and into catheter cannula 74.

While still gripping housing 14, plunger 12 can then be advanced relative to housing 14 by applying forward pressure with the palm or heel of the same hand without forcing the tip of needle 46 further into the patient. At the point where front section 34 of plunger 12 moves forwardly past collar 40, the holding force on spring 80 is released and retraction is initiated, thereby allowing spring 80 to expand, driving the rear end of needle assembly 18 rearwardly into plunger body 30 and retracting needle 46 inside housing 14. During this procedure, catheter hub seal 58 is held inside the rear of hub 66 by frictional engagement and, as needle 46 retracts through aperture 92 of hub seal 58, the retraction force is dampened by the slight frictional drag exerted on the outside surface of needle 46 by the thin elastomeric web. The slight frictional drag which also serves to wipe off any blood on the outside of the needle. Once needle 46 is retracted inside housing 14, aperture 92 contracts, and hub seal 58 reduces, limits, blocks or impedes the accidental discharge of any body fluid from the rear opening of hub 66 pending removal of the hub seal incidental to the attachment of hub 66 to another device. With catheter introducers that do not have a plunger and retraction spring, needle 46 is completely withdrawn by pulling housing 14 further backward relative to hub 66 and catheter hub seal 58.

Catheter hub seal 58 can perform several valuable functions when incorporated as part of catheter introducer 10 of the invention. Catheter hub seal 58 reduces the likelihood of unintentional separation of catheter assembly 16 from housing 14 during shipping and handling prior to use. Rearwardly facing recess 94 of hub seal 58 assures proper coaxial alignment between housing 14 and catheter hub assembly 16. Rearwardly projecting positioning tab 61 facilitates both proper coaxial and rotational alignment between hub seal 58 and housing 14 so that removal tab 60 projects upwardly and not at some other rotational position relative to housing 14. During insertion of needle 46 and catheter 74 into a blood vessel, catheter hub seal 58 helps stabilize hub 66, and tab 60 keeps the index finger of the user from contacting and possibly contaminating hub 66. During withdrawal of needle 46, the slight contact between the thin, preferably elastomeric, web of hub seal 58 and the outside surface of needle 46 wipes blood off the outside of needle 46. During retraction, contact between the thin web of hub seal 58 and the outside of needle 46 also dampens the retraction force to slow down the needle and thereby reduce the likelihood of forward splatter in response to retraction. Catheter hub seal 58 also impedes and reduces the likelihood of fluid spillage out of the catheter hub after withdrawal of needle 46 from hub 66 and hub seal 58. Removal tab 60 of hub seal 58 identifies the top (bevel) side of catheter introducer 10 and can be gripped for leverage during subsequent removal of hub seal 58 during from the opening at the back of hub 66.

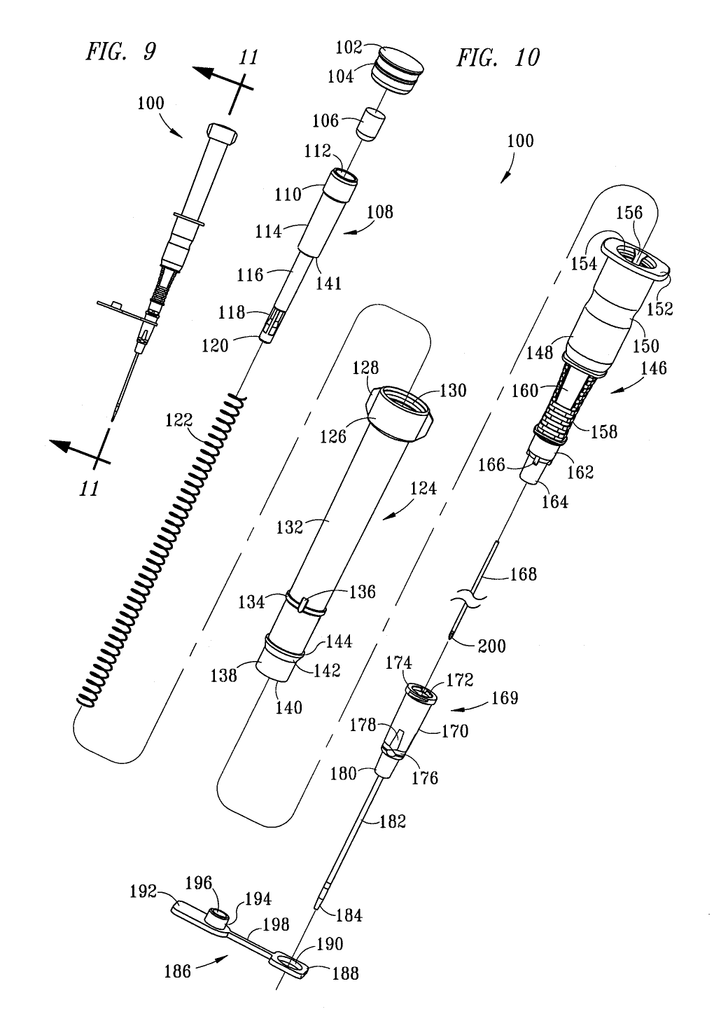

Another embodiment of the invention is further described and explained in relation to FIGS. 9-16. Referring first to FIGS. 9-11, IV catheter introducer 100 of the invention can comprise as principal elements plunger 124, housing 146, needle holder 108, needle 168, retraction spring 122, catheter hub assembly 169, hub seal device 186, and a removable cover (not shown) that is similar to cover 76 of FIGS. 1 and 2. Although a retractable needle is not required for use of hub seal device 186 of the invention, the use of a retractable needle can reduce the likelihood of inadvertent needle stick injuries once housing 146 is separated from catheter hub 170 and needle 168 is withdrawn from flexible cannula 182. Plunger assembly 124, housing 146, needle holder 108, and catheter hub assembly 169 can all be made of FDA-approved, moldable polymeric materials, although it will be understood that different parts can comprise different materials. Hub seal device 186 can be made of a polymeric material, including for example an elastomeric material such as rubber.

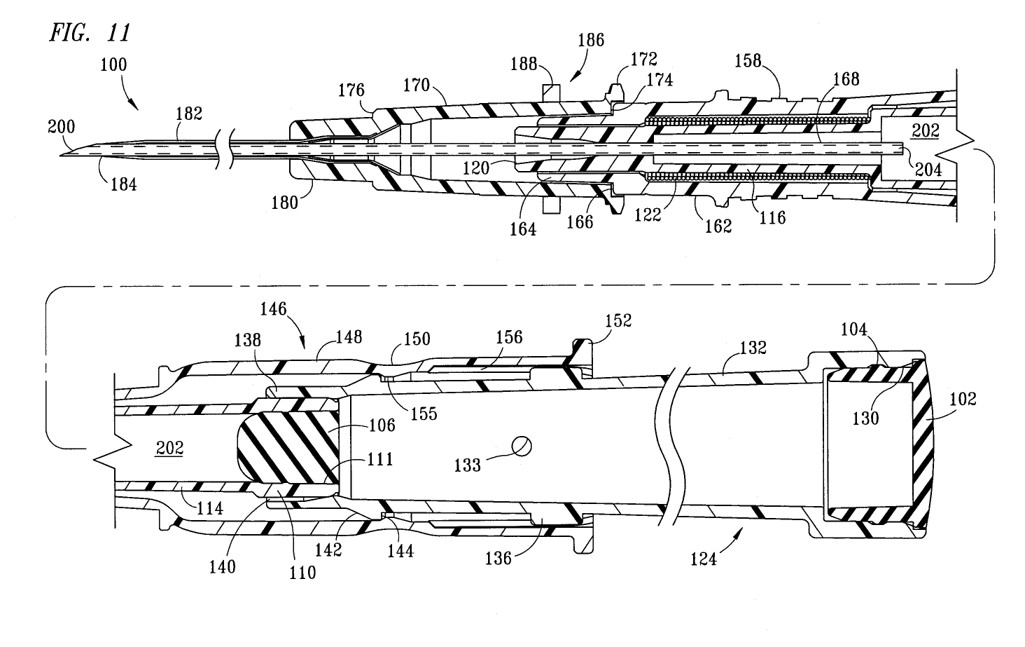

Plunger assembly 124 can comprise a substantially tubular body with three body sections 132, 134, 138. Plunger body sections 132 has an inside diameter that tapers inwardly from rear to front and a rear opening surrounded by collar 126. End cap 102 with one or more sealing rings 104 is desirably insertable into frictional engagement with the inside of collar 126 (more visible in FIG. 11), although other similarly effective cap configurations can likewise be used. The forwardly extending, smaller-diameter front section 138 of plunger assembly 124 can comprise a front end 140 having an opening (visible in FIG. 11) with a length an inside diameter sufficient to receive rearwardly facing end collar 110 of needle assembly 108. Vent hole 133 (FIG. 11) can be provided in plunger body section 132 to reduce the likelihood of blood splattering forwardly out of IV catheter introducer 100 during retraction.

Whereas needle 46 is depicted as already installed inside needle holder 19 in FIG. 2, needle 168 is depicted prior to installation in needle holder 108 in FIG. 10. It should be understood, however, that needle 168 as shown in FIG. 10 can be attached in fixed axial relation to needle holder 108 after needle holder 108 and retraction spring 122 are seated inside plunger 124. Referring to FIG. 11, opening 120 in the front end of needle holder 108 can be tapered to receive blunt end 204 of needle 168 through the front of needle holder 108 either prior to or subsequent to seating needle holder 108 inside housing 146. Because the forwardly extending tip of needle holder 108 protrudes forwardly from front section 164 of housing 146, needle 168 can be inserted into opening 120 from the front and cemented or otherwise secured in place without having to pass beveled end 200 through needle holder 108 and without any likelihood of occluding opening 204 at the rearwardly facing blunt end of needle 168 with cement.

As shown, needle holder 108 further comprises end collar 110 and forwardly extending, inwardly stepped sections 114, 116 separated by annular shoulder 141. Section 118 of needle holder 108 can comprise a coaxial longitudinal passageway sized to receive and hold a portion of the shaft of needle 168 in a fixed axial position relative to needle holder 108. Section 118 of needle holder 108 has an outside diameter small enough to be insertable into coiled retraction spring 122. Annular shoulder 141 has sufficient width to abut the top of retraction spring 122 when installed inside housing 146 and to limit the rearward expansion of spring 122 prior to retraction. (Although spring 122 is depicted in FIG. 10 as being disposed between needle holder 108 and plunger 124, it will be apparent to those of skill in the art upon reading this disclosure in relation to the drawings that the forward end of spring 122 seats inside housing 146 (FIG. 11), and will accordingly pass through plunger 124.)

Needle 168 can have a beveled front end, a blunt rear end, with the blunt end being insertable into, and held in fixed relation to, section 118 of needle holder 108 by any suitable means such as, for example, by a commercially available adhesive satisfactory for use in such applications. Such adhesive can be laser cured to facilitate high-speed manufacture. Needle 168 is preferably long enough to project forwardly from section 118 a distance sufficient to be clinically effective for insertion through catheter hub assembly 169, and to project rearwardly through the interior of sections 116, 114 and collar 110 so that blunt end 204 extends into flash chamber 202 (FIG. 11) inside body section 114.

Needle holder 108 can be unitarily molded out of substantially transparent plastic to facilitate viewing of liquid, such as blood, inside flash chamber 202 through window 160 (FIG. 10) in housing 146 during use of the device. This serves as a visual indicator to the user that needle 168 and catheter cannula 182 have been successfully inserted into a vessel. End collar 110 of needle holder 108 can have an outside diameter sized to slidably engage the inside surface of front section 138 of plunger 124 and an inside surface sized to frictionally engage plug 106.

During assembly of catheter introducer 100, plug 106 can be installed inside collar 110 prior to inserting collar 110 into front section 138 of plunger 124. A principal function of plug 106 is to retain a body liquid, such as blood, that enters flash chamber 202 inside stepped body section 114 of needle holder 108 after insertion of needle 168 and catheter cannula 182 into a vessel and prior to withdrawal of needle 168 from cannula 182. Both plug 106 and end cap 102 are disclosed in FIG. 11 as being made of a rubbery or elastomeric material to prevent the escape of any body fluid outside the back of needle holder 108 and plunger 124.

Housing 146 can comprise a centrally disposed, longitudinally extending tubular cavity that is coaxially aligned with the centrally disposed longitudinal cavities through plunger 124, needle holder 108, and catheter hub assembly 169. The inside diameter of housing 146 can be stepped inwardly to provide an annular seating surface for the forwardly extending end of spring 168 and an annular seating surface for annular shoulder 141 of needle holder 108 inside housing 146 (as seen in FIG. 11). Referring to FIG. 10, molded ridges 158 or other similarly effective surface texturing can be provided for use as finger grips and substantially flat wall section 160 can be provided as a viewing window into underlying flash chamber 202 of needle holder 108 as seen in FIG. 11. Housing 146 and needle holder 108 can be molded from clear plastic to facilitate viewing of blood flowing into flash chamber 202 from the blunt end of needle 168. One such plastic is polycarbonate.

Plunger 124 can have an annular rib 134 and circumferentially spaced, elongate keys 136 projecting outwardly from the outside surface of section 132 to guide plunger 124 as it advances forwardly into housing 146 during assembly. Keys 136 can be alignable with longitudinally directed keyways 156 (FIGS. 10-11) in the inside wall of housing 146 to maintain a preferred rotational alignment of plunger 124 and needle holder 108 inside housing 146. This rotational alignment can assist in assuring that bevel 200 of needle 168 is upwardly facing as plunger 124 is advanced forwardly relative to housing 146 during insertion of needle 168 into a vessel.

Referring again to FIGS. 10 and 11, forwardly facing inclined ramp 142 having a rearwardly facing, substantially square shoulder 144 is provided at the rear of section 138 plunger assembly 124. Ramp 142 slides past inwardly projecting annular rib 155 of housing 146 when plunger 124 is inserted into housing 14 during assembly of IV catheter introducer 100. Inwardly projecting annular rib 155 can be formed inwardly of annular groove 150 in outside wall 148 of housing 146. Abutting engagement between rearwardly facing shoulder 144 of ramp 142 and annular rib 155 subsequently limits rearward movement of plunger 124 relative to housing 146 sufficiently to prevent the forward end of plunger 124 from being accidentally or intentionally withdrawn from housing 146 prior to or following use of catheter introducer 100. Prior to use of catheter introducer 100, the abutting engagement also resists the rearwardly directed force of compressed spring 168 pushing against annular shoulder 141 of needle holder 108, which force is transmitted to plunger 124 from needle holder 108 through the engagement between collar 110 and the inside wall of section 138 in cooperation with inwardly facing annular ridge inside plunger 124 (FIG. 11). The inside diameter of that portion of housing 146 that is opposite section 118 of needle holder 108 can be slightly greater than the outside diameter of compressed spring 168 to accommodate the spring and to serve as an outer spring guide during compression as needle holder 108 is seated inside housing 146 when ramp 142 of plunger 124 is moved forwardly of annular rib 155 inside housing 146.

Referring to FIGS. 9-11, catheter hub assembly 169 can comprise tapered tubular hub 170 having a connector collar 172 on the rearwardly facing end. Connector collar 172 can be configured for easy attachment to cooperatively configured end connectors of conventional, commercially available IV infusion sets, blood collection bags, or other devices or accessories. Catheter cannula 182 is preferably a medically approved, flexible polymeric cannula having an inside diameter slightly greater than the outside diameter of needle 168 with a tapering outside wall near its distal end 184 and a length sufficient to cover all but the beveled end 200 of needle 168 when needle 168 is inserted through the catheter cannula. Catheter cannula 182 can be secured to the inside of tapered, forwardly extending tip 180 of catheter hub assembly 169 by any suitable means such as a press-fit ring disposed between the hub and catheter, adhesive, laser or ultrasonic welding, or the like. A slidable cover such as cover 76 in FIGS. 1 and 2, can be made of molded plastic with a length sufficient to protect the beveled tip of needle 168 prior to use and an inside diameter that frictionally engages the outside surface of tip 180 of catheter hub assembly 169.

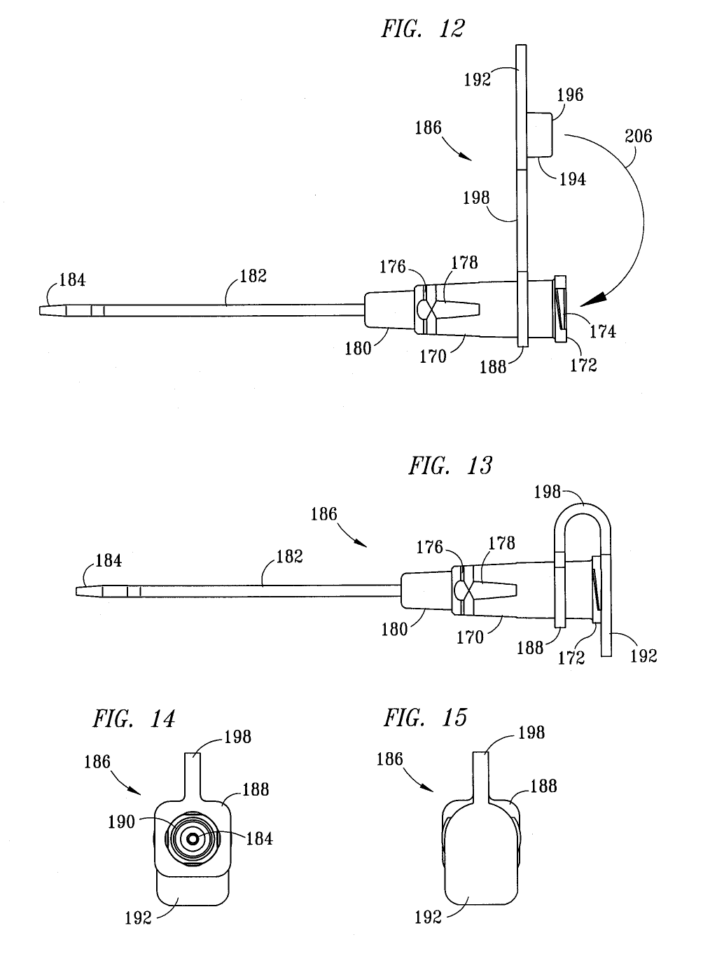

According to one embodiment of the invention, catheter seal device 186, as seen in FIGS. 9-15, can be made of rubber or a rubber-modified plastic material and can comprise an attachment means such as support ring 188 having an inwardly facing opening 190 such that it can be slipped onto and remain frictionally engaged with hub 170 of catheter hub assembly 169. Hub seal 196 can be an elastomeric plug having an outwardly facing, substantially cylindrical surface 194 that, together with removal tab 192, is unitarily molded together with support ring 188 and is joined to support ring 188 by a foldable hinge or tether 198 that is cooperatively configured to allow hub seal 196 to be inserted into the rearwardly facing opening of hub 170, as indicated by arrow 206 of FIG. 12 and by FIG. 13. Unlike with the embodiment previously described in relation to FIGS. 1-8, hub seal 196 is installed in the rear opening of catheter hub 170 following separation of housing 146 from catheter hub assembly 169 and withdrawal of needle 168 from catheter hub assembly 169 following insertion of flexible catheter cannula 182 into a bodily vessel. If desired, catheter seal device 186 (FIG. 10) can be attached or made attachable to catheter hub assembly 169 by frictional engagement between support ring 188 and hub 170, or by any other similarly effective means that is optionally provided such as, for example and without limitation, an attachment clip; adhesive; thermal, ultrasonic or chemical welding; or the like. The provision of some means or mechanism for attaching or releasably attaching catheter seal device 186 to catheter hub assembly 169 or to some part of housing 146 prior to use reduces the likelihood that hub seal 196 will be misplaced, dropped, or somehow contaminated prior to insertion of hub seal 196 into hub 170. It should also be appreciated, however, that it is within the scope of this embodiment of the invention to provide a catheter seal device 186 that is not attached or attachable to either hub 170 or to housing 146 prior to insertion of hub seal 196 into hub 170. A catheter seal device can, for example, be made, sterilized and wrapped separately from the other parts of catheter introducer 100, or be wrapped inside a chamber or compartment attached to a package in which one or more other portions of catheter introducer 100 are provided.

Referring to FIGS. 9-11, following assembly of catheter introducer 100, needle 168 projects forwardly from catheter cannula 182. Rear collar 110 of needle holder 108 can be frictionally engaged by the inside surface of section 138 of plunger assembly 12, and the escape of body liquid from the rear of flash chamber 202 is blocked by frictionally engaged plug 106. Retraction spring 122 can be compressed inside the longitudinally extending annular space between housing 146 and annular shoulder 141 of needle holder 108, and held in compression prior to retraction by contacting engagement between rear shoulder 144 of annular ramp 142 of plunger 124 (FIG. 2) and inwardly facing annular rib 155 opposite annular groove 150 of housing 146. Although retraction spring 168 is depicted as fully compressed in FIG. 11, it should be appreciated that the annular space into which spring 168 is compressed is desirably slightly longer than the fully compressed length of spring 168 to provide a slight tolerance during manufacture and assembly so that complete compression is not required in order for the spring to fit within the annular space.

At the time of use of IV catheter introducer 100, following removal of the needle cover, needle 168 and the forwardly extending tip of catheter cannula 182 are desirably inserted into the body, such as into a blood vessel, by lightly gripping ribs 158 (or other provided gripping surface) of housing 146 and applying forward pressure to housing 146 to advance needle 168 and catheter cannula 182 with the bevel of needle 168 facing upwardly. Once needle 168 punctures the wall of the vessel and catheter cannula 182 is properly positioned a sufficient distance inside the vessel, housing 146 is preferably pulled backward a short distance, such as from about 0.25 to about 0.5 inches, while applying downward pressure to catheter hub 170. This downward pressure prevents cannula 182 from withdrawing from the vessel as needle 168 moves rearwardly relative to catheter hub assembly 169 until separation of housing 146 from hub 170 is achieved.

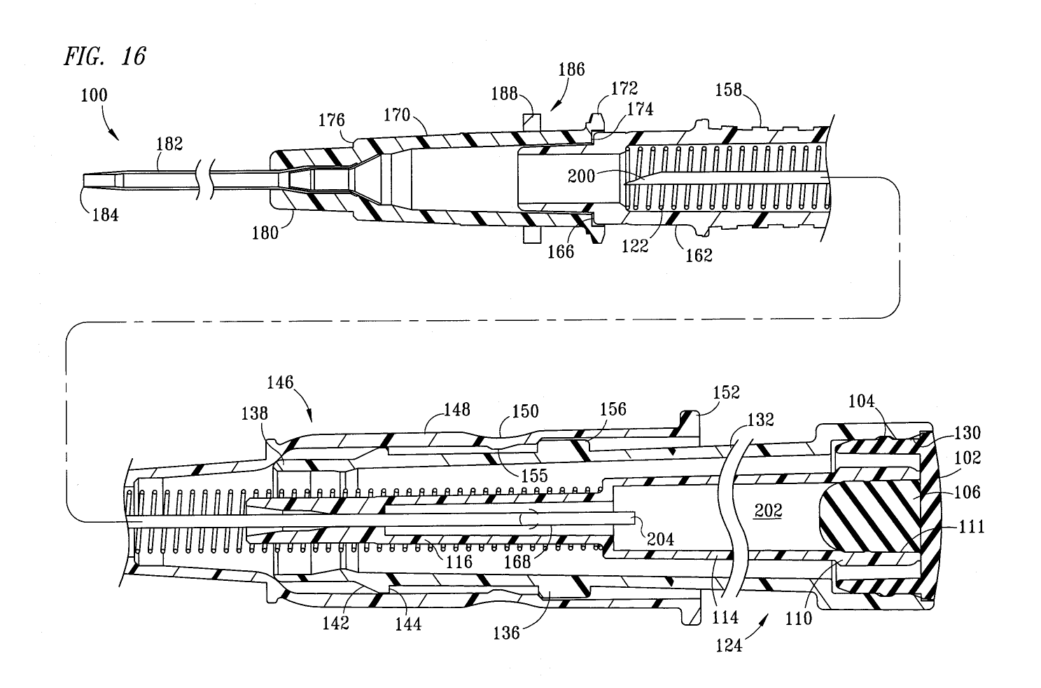

To initiate retraction, and referring to FIG. 16, while still gripping housing 146, plunger 124 can be advanced relative to housing 146 by applying forward pressure to cap 102 with the palm or heel of the same hand. At the point where front section 138 of plunger 124 moves forwardly past collar 110 of needle holder 108, the holding force on spring 122 is released and retraction is initiated, thereby allowing spring 122 to expand, driving the rear end of needle holder 108 rearwardly into plunger 124, and retracting beveled front tip 200 of needle 169 inside housing 146. The uncoiled length and resistance to re-compression of spring 122 are desirably such that spring 122 will continue to hold tip 184 of needle 168 inside housing 146 during handling subsequent to retraction.

Immediately following this procedure, hub seal 196 of catheter seal device 186 is desirably folded over and behind rear collar 172 of catheter hub 170 as indicated by arrow 206 in FIG. 12, and hub seal 196 is inserted into the opening defined by rear collar 172 to a position as shown in FIGS. 13-15, in which no portion of hub seal 196 is visible. Hub seal 196 is desirably configured and made so as to provide a fluid seal in the back of hub 170. At some later time, when it is desired to attach hub 170 to another device, hub seal 196 can be removed by pulling on tab 192 while catheter hub 170 is secured by manual pressure, tape or a combination thereof to prevent accidental or inadvertent premature removal of catheter cannula 182 from the bodily vessel into which it is inserted. Threads 174 can be provided outside of collar 172 to facilitate subsequent attachment of catheter hub 170 to another device as needed for the same patient.

Other alterations and modifications of the preferred embodiments described above will become apparent to those of ordinary skill in the art upon reading this disclosure, and it is intended that the scope of the invention disclosed herein be limited only by the broadest interpretation of the appended claims to which the inventors are legally entitled.

1. A catheter introducer comprising a housing, a catheter hub assembly releasably connected to the housing in coaxial alignment with the housing, a needle projecting from the housing through the catheter hub assembly, and a catheter hub seal releasably engageable with a rear opening of the catheter hub assembly to restrict fluid leakage from the catheter hub assembly following separation of the housing and withdrawal of the needle from the catheter hub assembly.

2. The catheter introducer of claim 1 wherein the catheter hub seal is disposed between and releasably connects the housing and the catheter hub assembly.

3. The catheter introducer of claim 2 wherein the needle penetrates the catheter hub seal.

4. The catheter introducer of claim 1 wherein the catheter hub seal releasably engages the rear opening of the catheter hub assembly.

5. The catheter introducer of claim 2 wherein the catheter hub seal releasably engages the rear opening of the catheter hub assembly.

6. The catheter introducer of claim 2 comprising a needle holder, wherein at least part of the needle holder is disposed inside the housing and wherein the catheter hub seal releasably engages the needle holder.

7. The catheter introducer of claim 1 comprising a needle holder, wherein at least part of the needle holder is disposed inside the housing.

8. The catheter introducer of claim 7 wherein a part of the needle holder projects forwardly from the housing.

9. The catheter introducer of claim 1 wherein the needle is retractable inside the housing.

10. The catheter introducer of claim 9 wherein the needle and at least part of the needle holder are retractable inside the housing.

11. The catheter introducer of claim 2 wherein the needle is retractable inside the housing.

12. The catheter introducer of claim 6 wherein the needle and at least part of the needle holder are retractable inside the housing.

13. The catheter introducer of claim 9, further comprising a retraction spring seated inside the housing.

14. The catheter introducer of claim 11, further comprising a retraction spring seated inside the housing.

15. The catheter introducer of claim 6, further comprising a plunger assembly releasably connected to the needle holder.

16. The catheter introducer of claim 7, further comprising a plunger assembly releasably connected to the needle holder.

17. The catheter introducer of claim 15 wherein the needle holder is releasable from the plunger assembly by forward movement of the plunger assembly relative to the housing.

18. The catheter introducer of claim 16 wherein the needle holder is releasable from the plunger assembly by forward movement of the plunger assembly relative to the housing.

19. The catheter introducer of claim 1 wherein the catheter hub seal comprises an elastomeric material.

20. The catheter introducer of claim 2 wherein the catheter hub seal comprises an elastomeric material.

21. The catheter introducer of claim 12 wherein the needle has a longitudinal axis and wherein the catheter hub seal comprises a thin elastomeric web that is substantially transverse to a longitudinal axis through the needle.

22. The catheter introducer of claim 1 wherein the catheter hub seal further comprises a removal tab.

23. The catheter introducer of claim 2 wherein the catheter hub seal further comprises a removal tab.

24. The catheter introducer of claim 14 wherein the catheter hub seal and removal tab are unitarily molded.

25. The catheter introducer of claim 1 wherein the catheter hub seal is connected to an outside surface of the catheter hub assembly.

26. The catheter introducer of claim 25 wherein the catheter hub seal is releasably connected to an outside surface of the catheter hub assembly.

27. The catheter introducer of claim 26 wherein the catheter hub seal is releasably connected to an outside surface of the catheter hub assembly by a flexible tether.

28. The catheter introducer of claim 27 wherein the flexible tether is foldable to a position where the catheter hub seal is removably engageable with the rear opening of the catheter hub assembly.

29. The catheter introducer of claim 6 wherein the needle holder further comprises a rear opening and a sealing plug disposed in the rear opening.

30. The catheter introducer of claim 7 wherein the needle holder further comprises a rear opening and a sealing plug disposed in the rear opening.

31. The catheter introducer of claim 1 wherein the catheter hub seal cooperates with a part of the housing to establish a preferred rotational alignment between the hub seal and housing.

32. The catheter introducer of claim 1 wherein the catheter assembly comprises a flexible catheter cannula.

33. The catheter introducer of claim 1 wherein the flexible catheter cannula is insertable into a blood vessel.

34. The catheter introducer of claim 33 wherein the catheter hub seal restricts blood flow out of the hub following withdrawal of the needle.

35. The catheter introducer of claim 1 wherein the catheter assembly comprises a pair of outwardly extending, opposed wings.

36. The catheter introducer of claim 1 comprising a needle retraction assembly.

37. The catheter introducer of claim 1, further comprising a plunger with a forwardly extending end portion that is slidably insertable into the housing.

38. An IV catheter introducer comprising a housing having a needle retraction assembly seated therein with a needle projecting forwardly from the housing and biased rearwardly prior to retraction, an IV catheter assembly having a forwardly extending flexible catheter attached to a rearwardly facing tubular hub, and a catheter hub seal disposed between the housing and hub.

39. The IV catheter introducer of claim 38, further comprising a releasable cover disposed over at least part of the needle and catheter.

40. The IV catheter introducer of claim 38 wherein the catheter hub seal comprises an elastomeric material.

41. The IV catheter introducer of claim 38 wherein the catheter hub seal comprises a forwardly projecting portion that frictionally engages the hub.

42. The IV catheter introducer of claim 38 wherein the catheter hub seal comprises a rearwardly facing recess that receives a portion of the needle retraction assembly.

43. The IV catheter introducer of claim 38 wherein the catheter hub seal comprises a thin web through which the needle is inserted.

44. The IV catheter introducer of claim 38 wherein the catheter hub seal comprises a first tab that projects outwardly from the housing and hub on a side of the introducer that corresponds to a side of the needle having a bevel.

45. The IV catheter introducer of claim 44 wherein the catheter hub seal comprises a second tab that projects toward the housing and cooperates with a part of the housing to establish a preferred rotational alignment between the hub seal and housing.

46. The IV catheter introducer of claim 38 wherein the IV catheter assembly comprises a pair of laterally extending wings.

47. The IV catheter introducer of claim 38 further comprising a plunger slidably insertable into an opening in a rear portion of the housing.

48. The IV catheter introducer of claim 47 wherein the plunger and housing cooperate to maintain a biasing force on the needle prior to retraction.

49. The IV catheter introducer of claim 38 wherein the forwardly projecting needle is biased rearwardly by a spring.

50. The IV catheter assembly of claim 38 wherein the web wipes the outside of the needle as the needle is withdrawn from the IV catheter assembly and catheter hub assembly following use.

51. The IV catheter assembly of claim 43 wherein the catheter hub seal impedes liquid flow rearwardly out of the hub following withdrawal of the needle from the catheter hub seal.

52. The IV catheter introducer of claim 46 wherein the wings are attached to the hub.
